(19) United States
(12) Patent Application Publication
(10) Pub. No.: US 2025/0253422 A1
(43) Pub. Date: Aug. 7, 2025

(54) HYDROGEN SULFIDE REMOVAL UNIT AND BATTERY PACK

(71) Applicant: TOYOTA JIDOSHA KABUSHIKI KAISHA, Toyota-shi (JP)

(72) Inventors: Yasumasa OGUMA, Nagoya-shi (JP); Shigeyuki Mori, Nagoya-shi (JP)

(73) Assignee: TOYOTA JIDOSHA KABUSHIKI KAISHA, Toyota-shi (JP)

(21) Appl. No.: 19/047,375

(22) Filed: Feb. 6, 2025

(30) Foreign Application Priority Data
Apr. 2, 2024 (JP) 2024-059767

(51) Int. Cl. H01M 10/52; B01D 53/04; B01D 53/30; H01M 50/358

(52) U.S. Cl. CPC H01M 10/52; B01D 53/0438; B01D 53/30; H01M 50/358; B01D 2257/304; B01D 2259/45

(57) ABSTRACT

The present disclosure provides a hydrogen sulfide removal unit with improved strength against stress in the vertical direction, and a battery pack comprising the hydrogen sulfide removal unit. the hydrogen sulfide removal unit 120 of the disclosure comprises a hydrogen sulfide removing agent 121, and a hydrogen sulfide removing agent container 122 housing the hydrogen sulfide removing agent. The hydrogen sulfide removing agent container has at least one baffle plate 122a. The baffle plate is disposed at an angle within 10° from the vertical direction so as to connect the top surface and bottom surface of the hydrogen sulfide removing agent container.

### FIELD

The present disclosure relates to a hydrogen sulfide removal unit and to a battery pack.

Various desulfurizers have been known conventionally.

### BACKGROUND

PTL 1, for example, discloses a desulfurizer that carries out desulfurization of an unreformed gas to be used in a fuel cell, the desulfurizer comprising a cylindrical container that is able to house a desulfurization catalyst, an inlet and outlet for the unreformed gas respectively provided at both ends in the axial direction of the container, and gas passage-forming means that forms a flow passage for the unreformed gas, so that unreformed gas that has flowed in through the inlet flows in the axial direction and flows out through the outlet, while moving back and forth between a first region and second region bordered by a surface including the axis in the interior space of the container, the gas passage-forming means being provided by a plurality of first partition plates (baffle plates) that partition at least the first region into the inlet side and the outlet side and a plurality of second partition plates that partition at least the second region into the inlet side and the outlet side, wherein each of the plurality of first partition plates and each of the plurality of second partition plates is disposed across a spacing between them in the axial direction, and the first and second partition plates are alternately disposed.

In other words, PTL 1 discloses gas passage-forming means which consists of partition plates (baffle plates) joined to either the top surface or bottom surface of the container.

### CITATION LIST

### Patent Literature

[PTL1] Japanese Unexamined Patent Publication No. 2007-273142

### SUMMARY

### Technical Problem

As disclosed in PTL 1, a desulfurizer is sometimes subjected to stress caused by vibration during transport, for example. When the desulfurizer is transported by a vehicle, for example, the amount of vibration in the vertical direction is considerable, suggesting that stress is more powerful grater on the desulfurizer in the vertical direction.

Incidentally, battery packs are known which have a battery and an outer container housing the battery. When the battery is a sulfide-based battery comprising a sulfide-based compound, the sulfide-based compound may react with water to form hydrogen sulfide. In this regard, it is conceivable to apply the construction of the desulfurizer disclosed in PTL 1 as a hydrogen sulfide removal unit for a battery pack.

A battery pack with a sulfide-based battery is sometimes installed in a vehicle. During movement of a vehicle having an installed battery pack with a sulfide-based battery and hydrogen sulfide removal unit, powerful stress may act on the hydrogen sulfide removal unit in the vertical direction, as mentioned above.

It is an object of the present disclosure to provide a hydrogen sulfide removal unit with improved strength against stress in the vertical direction, and a battery pack comprising the hydrogen sulfide removal unit.

### Solution to Problem

The present inventors have found that the aforementioned object can be achieved by the following means.

### Aspect 1

A hydrogen sulfide removal unit, comprising a hydrogen sulfide removing agent and a hydrogen sulfide removing agent container housing the hydrogen sulfide removing agent, wherein:

the hydrogen sulfide removing agent container has at least one baffle plate, and the baffle plate is disposed at an angle within 10° from the vertical direction so as to connect the top surface and bottom surface of the hydrogen sulfide removing agent container.

### Aspect 2

The hydrogen sulfide removal unit according to aspect 1, wherein:
the perimeter of the baffle plate is joined to the inner peripheral surface of the hydrogen sulfide removing agent container,
the baffle plate at least partially has a distribution port, and
the ratio of the area of the distribution port with respect to the total area of the baffle plate and the distribution port is 30% or lower.

### Aspect 3

The hydrogen sulfide removal unit according to aspect 2, wherein:
the hydrogen sulfide removing agent is disposed at least at the lower part inside the hydrogen sulfide removing agent container, and
the distribution port is provided at the lower part of the baffle plate.

### Aspect 4

The hydrogen sulfide removal unit according to any one of aspects 1 to 3, wherein the hydrogen sulfide removing agent container has a plurality of the baffle plates.

### Aspect 5

The hydrogen sulfide removal unit according to aspect 4, wherein a meandering flow passage is formed by the plurality of baffle plates.

### Aspect 6

The hydrogen sulfide removal unit according to any one of aspects 1 to 5, wherein the hydrogen sulfide removing agent container is square cylindrical or rounded square cylindrical.

### Aspect 7

The hydrogen sulfide removal unit according to aspect 6, wherein:
the hydrogen sulfide removing agent is disposed at least at the lower part inside the hydrogen sulfide removing agent container,
the distribution port is provided at the lower part of each baffle plate, and
the distribution port of the 2m+1-th baffle plate, where m is 0 or a positive integer, and the distribution port of the 2n-th baffle plate, where n is a positive integer, are provided so as to contact mutually different side surfaces among the pair of side surfaces of the hydrogen sulfide removing agent container, thereby forming the meandering flow passage.

### Aspect 8

The hydrogen sulfide removal unit according to any one of aspects 1 to 7, wherein each baffle plate has a thick part that is thicker than the other parts.

### Aspect 9

A battery pack comprising a sulfide-based battery, a hydrogen sulfide removal unit according to any one of aspects 1 to 8, and an outer container housing the sulfide-based battery and the hydrogen sulfide removing agent, wherein:
the outer container has an opening allowing communication between the inside and outside of the outer container, and
the hydrogen sulfide removal unit is disposed so that the inside of the outer container can communicate with the outside of the outer container through the hydrogen sulfide removal unit and the opening.

### Aspect 10

The battery pack according to aspect 9, wherein:
the battery pack further comprises a gas detection unit housed in the outer container,
the gas detection unit comprises a hydrogen sulfide adsorbent that adsorbs hydrogen sulfide and releases the hydrogen sulfide and/or sulfur-containing gas derived from the hydrogen sulfide at or above a predetermined temperature, and a heating section that heats the hydrogen sulfide adsorbent, and a gas detection section that detects the hydrogen sulfide and/or sulfur-containing gas released from the hydrogen sulfide adsorbent, and
the gas detection unit and the hydrogen sulfide removal unit are disposed so that the inside of the outer container can communicate with the outside of the battery pack through the gas detection unit, the hydrogen sulfide removal unit and the opening.

### Advantageous Effects of Invention

According to the present disclosure it is possible to provide a hydrogen sulfide removal unit with improved strength against stress in the vertical direction, and a battery pack comprising the hydrogen sulfide removal unit.

### BRIEF DESCRIPTION OF DRAWINGS

FIG. 1 is a simplified perspective view showing an example of the hydrogen sulfide removal unit of the disclosure.

FIG. 2 is a simplified perspective view showing an example of the hydrogen sulfide removal unit of the disclosure.

FIG. 3 is a simplified perspective view showing an example of the hydrogen sulfide removal unit of the disclosure.

FIG. 4 is a simplified perspective view showing an example of the hydrogen sulfide removal unit of the disclosure.

FIG. 5 is a simplified perspective view showing an example of the hydrogen sulfide removal unit of the disclosure.

FIG. 6 is a schematic plan view showing an example of the hydrogen sulfide removal unit of the disclosure.

FIG. 7 is a schematic diagram showing an example of a battery pack of the disclosure.

FIG. 8 is a schematic diagram showing an example of a battery pack of the disclosure.

### DESCRIPTION OF EMBODIMENTS

An embodiment of the disclosure will now be described in detail. The disclosure is not limited to the embodiment described below, however, and various modifications may be implemented which do not depart from the gist thereof.

### Hydrogen Sulfide Removal Unit

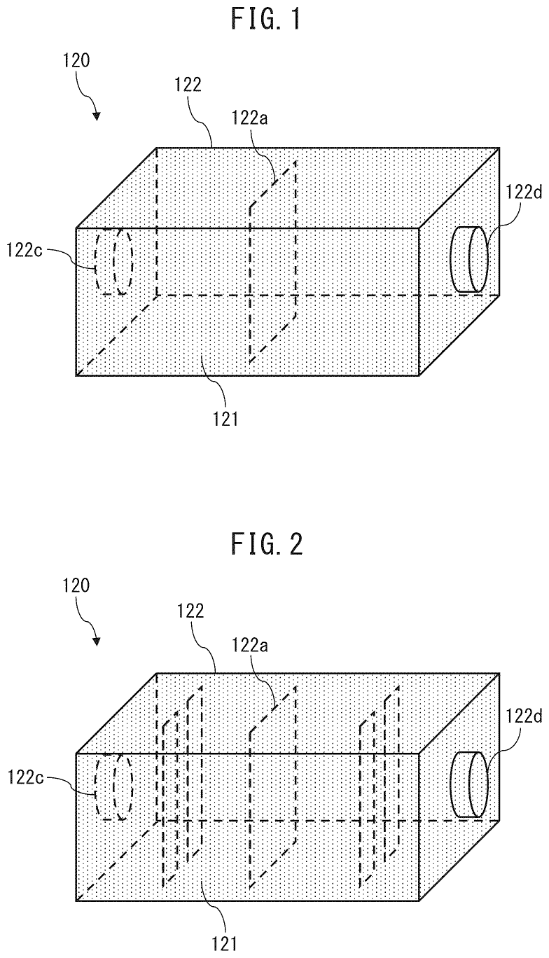

As illustrated in FIGS. 1 to 6, the hydrogen sulfide removal unit 120 of the disclosure comprises a hydrogen sulfide removing agent 121, and a hydrogen sulfide removing agent container 122 housing the hydrogen sulfide removing agent 121. The hydrogen sulfide removing agent container 122 has at least one baffle plate 122a. The baffle plate 122a is disposed at an angle within 10° from the vertical direction so as to connect the top surface and bottom surface of the hydrogen sulfide removing agent container 122.

The present inventors have found that if the baffle plate 122a in a hydrogen sulfide removing agent container 122 is disposed at an angle within 10° from the vertical direction so as to connect the top surface and bottom surface of the hydrogen sulfide removing agent container 122, then it is possible to improve the strength against stress in the vertical direction of the hydrogen sulfide removal unit 120. Such a construction can also lengthen the flow passage for hydrogen sulfide in the hydrogen sulfide removing agent container 122, thus allowing more effective removal of hydrogen sulfide.

The elements composing the hydrogen sulfide removal unit of the disclosure will now be described.

### <Hydrogen Sulfide Removing Agent>

As illustrated in FIGS. 1 to 6, the hydrogen sulfide removal unit 120 of the disclosure comprises a hydrogen sulfide removing agent 121. The hydrogen sulfide removing agent 121 has the function of removing hydrogen sulfide. For example, when the hydrogen sulfide removal unit 120 is housed together with the sulfide-based battery in the outer container of the battery pack, the hydrogen sulfide removed by the hydrogen sulfide removing agent 121 may be hydrogen sulfide generated by reaction between the sulfide-based compound and moisture in the sulfide-based battery. Examples of moisture that reacts with sulfide-based compounds include moisture in external air that has infiltrated into the outer container, and water that has infiltrated into the outer container.

The hydrogen sulfide removing agent is not particularly restricted, and for example, it may be an agent that removes hydrogen sulfide by chemical or physical adsorption of hydrogen sulfide.

When the hydrogen sulfide removing agent is an agent that chemically adsorbs hydrogen sulfide, the hydrogen sulfide removing agent may be an alkaline substance. Examples of alkaline substances include hydroxides of alkali metals or alkaline earth metals, and specifically NaOH, KOH, Mg(OH)₂ and Ca(OH)₂. In this case, the hydrogen sulfide removing agent may be encapsulated in gas-permeable microcapsules or mixed with a resin and molded into a sheet, for example.

When the hydrogen sulfide removing agent is an agent that physically adsorbs hydrogen sulfide, the hydrogen sulfide removing agent may be activated carbon or a silica gel, for example. These hydrogen sulfide removing agents, in powdered form, may also be used after solidification into pellets.

Examples of hydrogen sulfide removing agents include metals such as aluminum, manganese, copper, cobalt, zinc and nickel, and their metal oxides. These hydrogen sulfide removing agents may also be supported on a carrier. Zeolite is an example of a carrier.

The hydrogen sulfide removing agent may also be filled in so as to block the hydrogen sulfide flow passages. That is, the hydrogen sulfide removing agent may be filled throughout all of the flow passages. Such a construction will facilitate contact between the hydrogen sulfide and the hydrogen sulfide removing agent. From the viewpoint of gas permeability, the hydrogen sulfide removing agent in this case may be a powdered hydrogen sulfide removing agent solidified into pellets.

### <Hydrogen Sulfide Removing Agent Container>

As illustrated in FIGS. 1 to 6, the hydrogen sulfide removal unit 120 of the disclosure comprises a hydrogen sulfide removing agent container 122 housing the hydrogen sulfide removing agent 121.

The shape of the hydrogen sulfide removing agent container is not particularly restricted and may be cylindrical or polygonal cylindrical, for example. The hydrogen sulfide removing agent container may have articles with such shapes disposed, with the axial direction of the cylindrical body oriented horizontally. When the shape of the hydrogen sulfide removing agent container is cylindrical, it may be regular cylindrical or elliptical cylindrical. When the shape of the hydrogen sulfide removing agent container is polygonal cylindrical, the number of angles is not particularly restricted. The angular shapes of a polygonal cylindrical body may also be rounded. When the shape of the hydrogen sulfide removing agent container is polygonal cylindrical, it is most preferably square cylindrical or rounded square cylindrical.

The size of the hydrogen sulfide removing agent container is not particularly restricted and may be designed as appropriate in consideration of the amount of hydrogen sulfide to be generated and the size of the baffle plate 122a as described below.

The material of the hydrogen sulfide removing agent container is not particularly restricted so long as it is a material that can withstand corrosion by hydrogen sulfide. Such materials include metals such as aluminum and stainless steel.

### (Baffle Plate)

As illustrated in FIGS. 1 to 6, the hydrogen sulfide removing agent container 122 has at least one baffle plate 122a. The hydrogen sulfide removing agent container 122 may have one baffle plate 122a, as illustrated in FIGS. 1 and 4. The hydrogen sulfide removing agent container 122 may also have a plurality of baffle plates 122a, as illustrated in FIGS. 2, 3, 5 and 6, for example. When the hydrogen sulfide removing agent container 122 has a plurality of baffle plates 122a, the number of baffle plates 122a is not particularly restricted and may be 2 or more, 3 or more, 4 or more or 5 or more, and 10 or less, 8 or less, 7 or less, 6 or less or 5 or less, for example.

The baffle plate 122a is disposed at an angle within 10° from the vertical direction so as to connect the top surface and bottom surface of the hydrogen sulfide removing agent container 122, as illustrated in FIGS. 1 to 6. The baffle plate 122a may be disposed particularly at an angle within 7°, within 5°, within 3° or within 1° from the vertical direction. A small angle can more effectively improve the strength of the hydrogen sulfide removing agent container against stress in the vertical direction. The angle referred to here is the angle from the vertical direction in either the positive or negative direction. That is, the angle is within ±10°, within ±7°, within ±5°, within ±3° or within ±1° of the vertical direction. When a plurality of baffle plates are used, the inclination angles and inclining directions of each of the baffle plates may be the same or different.

For the purpose of the disclosure, the term “top surface” of the hydrogen sulfide removing agent container, when the hydrogen sulfide removing agent container is cylindrical, means the region where the circumferential angle is 120° or smaller in the circumference centered around the top of the hydrogen sulfide removing agent container. The “bottom surface” in this case means the region where the circumferential angle is 120° or smaller in the circumference centered around the bottom of the hydrogen sulfide removing agent container.

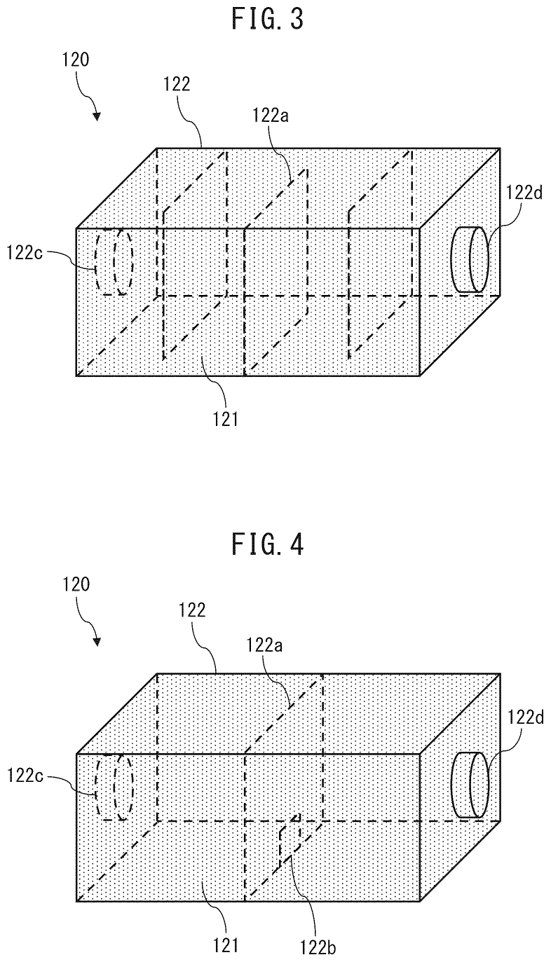

A meandering flow passage may be formed by the plurality of baffle plates 122a, as illustrated in FIG. 3. For example, in the hydrogen sulfide removing agent container, at least one first baffle plate running from a first side surface along the top surface and bottom surface, and at least partially not connected to the second side surface opposite the first side surface, and at least one second baffle plate running from the second side surface along the top surface and bottom surface, and at least partially not connected to the first side surface, may be disposed alternately in the direction of flow of hydrogen sulfide, to form a meandering flow passage. For the purpose of the disclosure, the “first side surface” and “second side surface”, when the shape of the hydrogen sulfide removing agent container is cylindrical, are the mutually opposing sides other than the “top surface” and “bottom surface”, and which are parallel to the direction of flow of the hydrogen sulfide.

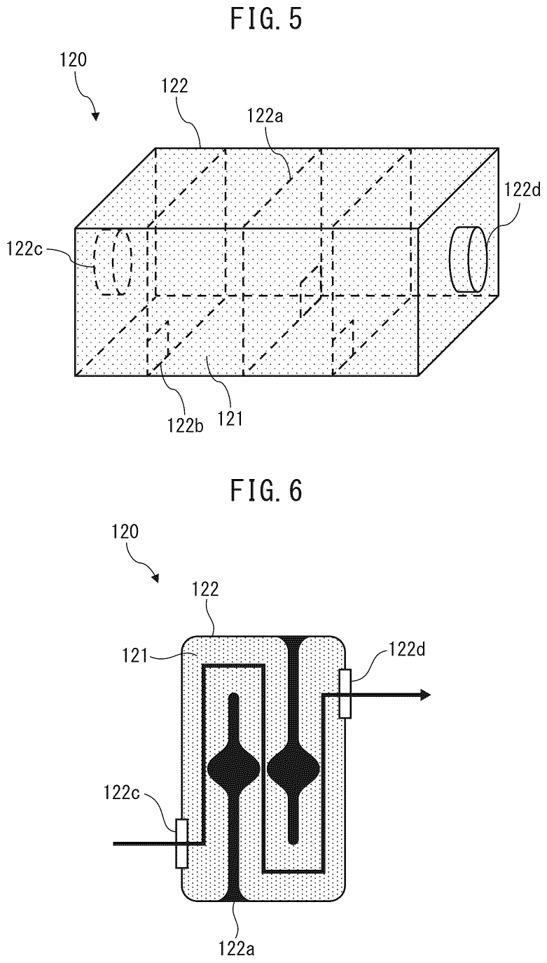

As illustrated in FIGS. 4 and 5, the perimeter of each baffle plate 122a may be joined to the inner peripheral surface of the hydrogen sulfide removing agent container 122, in which case the baffle plate 122a may at least partially have a distribution port 122b.

The size of the distribution port is not particularly restricted and may be designed as appropriate for the amount of hydrogen sulfide to be generated. For example, the size of the distribution port can be represented as the ratio of the area of the distribution port with respect to the total area of the baffle plate and the distribution port. The ratio of the area of the distribution port with respect to the total area of the baffle plate and distribution port may be 30% or lower. The ratio may be 1% or higher, 5% or higher, 10% or higher, 15% or higher or 20% or higher, and 30% or lower, 25% or lower, 20% or lower, 15% or lower or 10% or lower. Such a construction allows hydrogen sulfide to be more effectively removed.

As illustrated in FIGS. 4 and 5, the hydrogen sulfide removing agent 121 may be disposed at least at the lower part inside the hydrogen sulfide removing agent container 122, and the distribution port 122b may be provided at the lower part of the baffle plate 122a. Since the specific gravity of the hydrogen sulfide is greater than the specific gravity of air, disposing the hydrogen sulfide removing agent 121 at the lower part inside the hydrogen sulfide removing agent container 122 facilitates contact between the hydrogen sulfide and the hydrogen sulfide removing agent 121, thus allowing hydrogen sulfide to be removed more effectively. Vibration and impact can cause micronization of the hydrogen sulfide removing agent 121, often resulting in reduced bulk volume of the hydrogen sulfide removing agent 121. As a result, even if the hydrogen sulfide removing agent 121 is filling the entirety of the flow passages, regions with no hydrogen sulfide removing agent 121 present may form at the top of the hydrogen sulfide removing agent container 122. By providing the distribution port 122b at the lower part of the baffle plate 122a, therefore, hydrogen sulfide can pass through the distribution port 122b, via the hydrogen sulfide removing agent 121.

As mentioned above, the shape of the hydrogen sulfide removing agent container 122 may be polygonal cylindrical, in which case it is preferably square cylindrical or rounded square cylindrical. When the shape of the hydrogen sulfide removing agent container 122 is square cylindrical, as illustrated in FIG. 5, the hydrogen sulfide removing agent 121 may be disposed at least at the lower part inside the hydrogen sulfide removing agent container with the distribution port 122b provided at the lower part of the baffle plate 122a, and the distribution port 122b of the 2m+1-th baffle plate 122a, where m is 0 or a positive integer, and the distribution port 122b of the 2n-th baffle plate 122a, where n is a positive integer, may be provided so as to contact mutually different side surfaces among the pair of side surfaces of the hydrogen sulfide removing agent container, thereby forming a meandering flow passage. The value of m is not particularly restricted and may be 0, or a positive integer of 10 or less, for example. The value of n is also not particularly restricted and may be a positive integer of 10 or less, for example. Such a construction can also lengthen the flow passage for hydrogen sulfide at the lower part inside the hydrogen sulfide removing agent container, thus allowing more effective removal of hydrogen sulfide.

The shape of the distribution port is not particularly restricted and may be circular, elliptical or rectangular, for example. When the hydrogen sulfide removing agent container has a plurality of baffle plates, the shape of the distribution port of each baffle plate may be the same or different.

The number of distribution ports is not particularly restricted, and may be one or more, for example. When the hydrogen sulfide removing agent container has a plurality of baffle plates, the number of distribution ports of each baffle plate may also be the same or different. The number of distribution ports may be one, from the viewpoint of facilitating production.

The thickness of the baffle plate is not particularly restricted and may be designed as appropriate in consideration of the strength of the hydrogen sulfide removal container and the amount of hydrogen sulfide to be generated. When a plurality of baffle plates are used, the thicknesses of each of the baffle plates may be the same or different.

The thicknesses of each of the baffle plates may also differ within the same baffle plate, and for example, each baffle plate may have a thick part that is thicker than the other parts. Such a construction allows the strength of the hydrogen sulfide removing agent container to be more effectively increased. When the number of baffle plates is more than one, the number of baffle plates having thick parts that are thicker than the other parts may be one or more than one. When the number of baffle plates is more than one, the thicknesses of the parts matching the locations in the vertical direction among the mutually opposing baffle plates may be thickened, as illustrated in FIG. 6. With such a construction it is possible to help prevent hydrogen sulfide from circulating through the flow passage by pressure loss, thus allowing more effective removal of hydrogen sulfide. The arrows in FIG. 6 indicate the direction of flow of hydrogen sulfide.

The shapes of the baffle plates are not particularly restricted so long as the baffle plates are disposed at an angle within 10° from the vertical direction. For example, the shapes of the baffle plates may be planar shapes or screws, although they are preferably planar shapes from the viewpoint of facilitating production. When a plurality of baffle plates are used, the shapes of each of the baffle plates may be the same or different.

The sizes of the baffle plates are not particularly restricted and may be designed as appropriate for the amount of hydrogen sulfide to be generated. When a plurality of baffle plates are used, the sizes of each of the baffle plates may be the same or different.

The orientations of the baffle plates are also not particularly restricted. For example, each baffle plate may be disposed vertically with respect to the direction of flow of the hydrogen sulfide, or it may be disposed with a predetermined angle with respect to the direction of flow of the hydrogen sulfide. When the baffle plates are each disposed with a predetermined angle with respect to the direction of flow of the hydrogen sulfide, the predetermined angle may be an acute angle or an obtuse angle. From the viewpoint of lengthening the flow passage of hydrogen sulfide, the predetermined angle may be an acute angle near 90°, or each baffle plate may be disposed vertically with respect to the direction of flow of the hydrogen sulfide. When a plurality of baffle plates are used, the orientations of each of the baffle plates may be the same or different.

The material of the baffle plates is not particularly restricted so long as it is a material that can withstand corrosion by hydrogen sulfide. Such materials include metals such as aluminum and stainless steel.

The manner in which the baffle plates are arranged is not limited to the manner illustrated in FIGS. 1 to 6.

### (Inlet and Outlet)

The hydrogen sulfide removing agent container may be provided with an inlet 122c and outlet 122d for hydrogen sulfide. The positions, shapes and sizes of the inlet and outlet are not particularly restricted and may be designed as appropriate in consideration of removal efficiency for hydrogen sulfide.

### Battery Pack

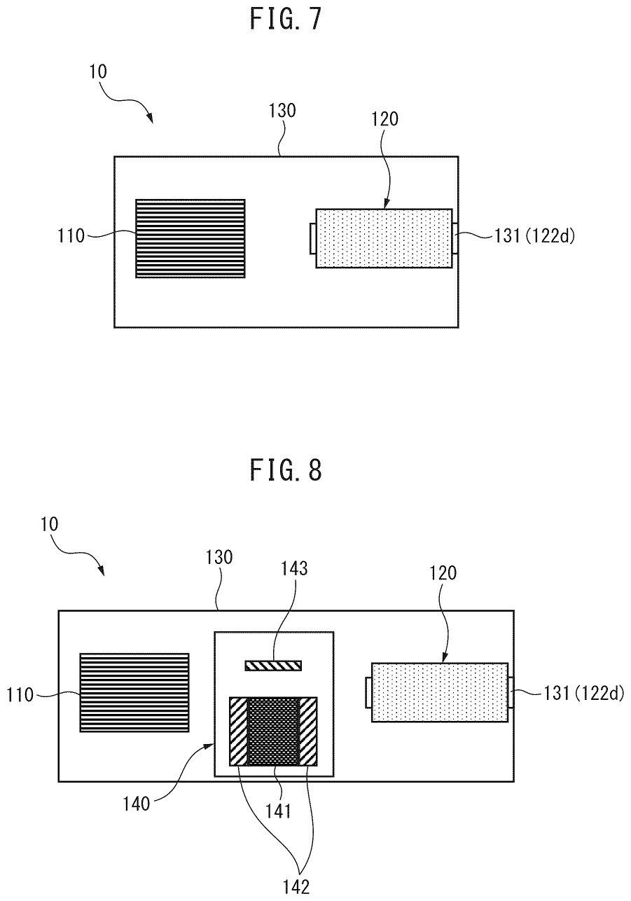

As illustrated in FIG. 7, the battery pack 10 of the disclosure comprises a sulfide-based battery 110, a hydrogen sulfide removal unit 120 of the disclosure, and an outer container 130 housing the sulfide-based battery and hydrogen sulfide removing agent. The outer container has an opening 131 allowing communication between the inside and outside of the outer container. The hydrogen sulfide removal unit is disposed so that the inside of the outer container can communicate with the outside of the outer container through the hydrogen sulfide removal unit and the opening.

The battery pack is often subjected to stress by vibration, depending on where it is installed. When a battery pack is installed in a vehicle, particularly strong stress acts on the battery pack in the vertical direction. Since the hydrogen sulfide removal unit the disclosure has improved strength against stress in the vertical direction, it is suitable as a hydrogen sulfide removal unit to be used in a such a battery pack.

The elements composing the battery pack of the disclosure will now be described.

### <Sulfide-Based Battery>

As illustrated in FIG. 7, the battery pack 10 of the disclosure comprises a sulfide-based battery 110. The term “sulfide-based battery” as used herein means a battery that includes a sulfide-based compound. The sulfide-based compound is not particularly restricted, and may be a sulfide-based solid electrolyte, for example. The sulfide-based battery 110 will sometimes generate hydrogen sulfide due to reaction between the sulfide-based compound and moisture.

The sulfide-based battery may be a liquid battery or a solid-state battery. The sulfide-based battery may also include a sulfide-based compound such as a sulfide solid electrolyte in one or more layers selected from among a positive electrode active material layer, an electrolyte layer and a negative electrode active material layer. The sulfide-based battery is most preferably a sulfide solid-state battery comprising a sulfide solid electrolyte in the solid electrolyte layer.

The term “solid-state battery” as used herein refers to a battery using at least a solid electrolyte as the electrolyte, and the solid-state battery may employ a combination of a solid electrolyte and a liquid electrolyte as the electrolyte. The solid-state battery of the disclosure may also be an all-solid-state battery, i.e. a battery employing only a solid electrolyte as the electrolyte.

The sulfide-based battery may be a primary battery, or a secondary battery such as a lithium ion battery or sodium ion battery.

When the sulfide-based battery of the disclosure is a lithium ion battery, examples of sulfide solid electrolytes may include, but are not limited to, sulfide amorphous solid electrolytes, sulfide crystalline solid electrolytes and argyrodite solid electrolytes. Specific examples of sulfide solid electrolytes include, but are not limited to, Li₂S—P₂S₅ (Li₇₃S₁₁, Li₃PS₄, Li₈P₂S₉), Li₂S—SiS₂, LiI—Li₂S—SiS₂, LiI—Li₂S—P₂S₅, LiI—LiBr—Li₂S—P₂S₅, Li₂S—P₂S₅—GeS₂ (Li₁₃GeP₃S₁₆, Li₁₀GeP₂S₁₂), LiI—Li₂S—P₂O₅, LiI—Li₃PO₄—P₂S₅ and Li₇₋ₛPS₆₋ₓClₓ, as well as combinations thereof.

When the sulfide-based battery of the disclosure is a sodium ion battery, examples of sulfide solid electrolytes may include, but are not limited to, Na₂S—P₂S₅ such as Na₃PS₄, or Na₃SbS₄ and Na_{2.88}Sb_{0.88}W_{0.12}S₄, or their combinations.

A sulfide solid electrolyte may be glass or crystallized glass (glass ceramic).

According to the disclosure, the sulfide-based battery may be a “laminated battery” (also known as “pouch battery”), as a battery having an electrode stack with a negative electrode collector layer, a negative electrode active material layer, an electrolyte layer, a positive electrode active material layer and a positive electrode collector layer stacked in that order, housed inside a laminate film as the battery-housing case. When the sulfide-based battery is a laminated battery, opening of the sealed sections of the laminate film can occur, allowing contact between the sulfur-based compound and moisture, and resulting in hydrogen sulfide generation from the sulfide-based battery. The sulfide-based battery of the disclosure is therefore particularly useful as a laminated battery.

The case housing the sulfide-based battery does not need to be a laminate film. That is, the sulfide-based battery may be a battery other than a laminated battery, such as a square, cylindrical or coin battery.

The materials for the negative electrode collector layer, negative electrode active material layer, electrolyte layer, positive electrode active material layer and positive electrode collector layer may appropriate and common materials used for such layers in a sulfide-based battery.

The shape and size of the electrode stack is not particularly restricted.

The shape, size and material of the case housing the battery are also not particularly restricted.

The sulfide-based battery housed in an outer container as described below is electrically connected in a serial or parallel manner, or in a combination of serial and parallel. The number of sulfide-based batteries housed in the outer container may be one. The sulfide-based battery may also be a battery module composed of two or more unit cells. In this case, the unit cells in each battery may be electrically connected in a serial manner or parallel manner, or in a combination of serial and parallel. The placement of the battery or batteries in the outer container is not particularly restricted and may be any desired placement. A plurality of batteries may be stacked together or disposed separately from each other.

The method for producing the sulfide-based battery is not particularly restricted, and it may be produced by a conventional publicly known method.

### <Hydrogen Sulfide Removal Unit>

As illustrated in FIG. 7, the battery pack 10 of the disclosure comprises a hydrogen sulfide removal unit 120. The hydrogen sulfide removal unit in the battery pack of the disclosure removes hydrogen sulfide which can be generated from the sulfide-based battery. The hydrogen sulfide removal unit will be understood by referring to the hydrogen sulfide removal unit of the disclosure as described above.

### <Outer Container>

As illustrated in FIG. 7, the battery pack 10 of the disclosure comprises an outer container 130. The outer container 130 houses the sulfide-based battery 110 and the hydrogen sulfide removal unit 120.

The shape and size of the outer container are not particularly restricted so long as they allow it to house the sulfide-based battery and hydrogen sulfide removing agent.

The material forming the outer container is also not particularly restricted and may be one commonly used for outer containers of battery packs. Such materials include metals, and specifically aluminum and stainless steel.

### (Opening)

As illustrated in FIG. 7, the outer container 130 has an opening 131 that allows communication between the inside and outside of the outer container 130. That is, the inside and outside of the outer container 130 communicate by the opening 131.

The inside of the outer container 130 has the hydrogen sulfide removal unit 120 disposed so as to allow communication with the outside of the outer container 130 via the hydrogen sulfide removal unit 120 and opening 131. That is, the hydrogen sulfide removing agent 121 is disposed so that even when hydrogen sulfide has been generated from the sulfide-based battery 110, hydrogen sulfide-containing gas is discharged out of the outer container 130 via the hydrogen sulfide removing agent 121. Such a construction allows hydrogen sulfide generated in the battery pack to be effectively removed.

The hydrogen sulfide removing agent container 122 in the battery pack 10 of the disclosure may also be connected to the opening 131. If the hydrogen sulfide removing agent container 122 is connected to the opening 131, gas containing hydrogen sulfide generated from the sulfide-based battery will be easier to discharge out of the outer container via the hydrogen sulfide removing agent. The connected portion between the hydrogen sulfide removing agent container and the opening is most preferably sealed.

The opening may also be provided with a ventilation film. As used herein, “ventilation film” means a film which is gas permeable but liquid water impermeable. If the opening is provided with a ventilation film it will be easier to inhibit infiltration of moisture (liquid water) from outside the battery pack.

The material of the ventilation film is not particularly restricted so long as it can function as a ventilation film. The material of the ventilation film may be stretched polytetrafluoroethylene or a nylon mesh, for example.

The opening may also be provided with a check valve. For the purpose of the disclosure, a check valve is a valve that can open outward from the outer container but cannot open inward into the outer container. The check valve may be open when the internal pressure of the outer container increases due to generation of hydrogen sulfide from the sulfide-based battery. Providing a check valve for the opening can effectively inhibit infiltration of moisture from outside the battery pack.

The type of check valve is not particularly restricted. For example, it may be an arm type, poppet type, swing type, wafer type, lift type, ball type or foot type check valve.

The members provided in the opening to inhibit infiltration of moisture are not limited to a ventilation film and check valve. The opening may also be provided with a member other than members for inhibiting infiltration of moisture.

The opening may also be provided with an exhaust part, for example. Such a construction allows hydrogen sulfide generated in the battery pack to be effectively removed. The exhaust part is not particularly restricted so long as it allows gas in the outer container to be emitted out of the outer container, and it may be an exhaust fan, for example. The exhaust part may be provided inside the outer container.

The shape of the opening is not particularly restricted and may be circular, elliptical, rectangular or linear, for example. A circular, elliptical or rectangular shape of the opening will facilitate attachment of the ventilation film or check valve members. A linear shape of the opening will help to inhibit infiltration of moisture from outside the battery pack even without members for inhibiting infiltration of moisture.

The number of openings is also not particularly restricted so long as the openings are constructed so that even when hydrogen sulfide has been generated from the sulfide-based battery, the hydrogen sulfide-containing gas is emitted out of the outer container via the hydrogen sulfide removing agent.

In the battery pack of the disclosure there is no need for any opening other than the openings described above, in order to allow communication between the inside and outside of the outer container. Such a construction allows hydrogen sulfide-containing gas to be effectively emitted out of the outer container via the hydrogen sulfide removing agent.

### <Gas Detection Unit>

As illustrated in FIG. 8, the battery pack 10 of the disclosure further comprises a gas detection unit 140 housed inside the outer container 130. The gas detection unit may comprise a hydrogen sulfide adsorbent 141 that releases hydrogen sulfide and/or sulfur-containing gas derived from hydrogen sulfide at or above a predetermined temperature, a heating section 142 that heats the hydrogen sulfide adsorbent, and a gas detection section 143 that detects hydrogen sulfide and/or sulfur-containing gas released from the hydrogen sulfide adsorbent.

That is, the battery pack of the disclosure has a sulfide-based battery, a gas detection unit, a hydrogen sulfide removal unit of the disclosure, and an outer container housing the sulfide-based battery, gas detection unit and hydrogen sulfide removal unit. The gas detection unit may also have a hydrogen sulfide adsorbent that adsorbs hydrogen sulfide and releases hydrogen sulfide and/or sulfur-containing gas derived from hydrogen sulfide at or above a predetermined temperature, a heating section that heats the hydrogen sulfide adsorbent, and a gas detection section that detects hydrogen sulfide and/or sulfur-containing gas released from the hydrogen sulfide adsorbent. The outer container of the disclosure comprises an opening allowing communication between the inside and outside of the outer container. The gas detection unit and the hydrogen sulfide removal unit are disposed so that the inside of the outer container can communicate with the outside of the battery pack through the gas detection unit, the hydrogen sulfide removal unit and the opening.

### (Hydrogen Sulfide Adsorbent)

As illustrated in FIG. 8, the gas detection unit 140 may comprise a hydrogen sulfide adsorbent 141 that releases hydrogen sulfide and/or sulfur-containing gas derived from hydrogen sulfide, at or above a predetermined temperature. Activated carbon is an example of such a hydrogen sulfide adsorbent. If the hydrogen sulfide adsorbent is activated carbon then it will be possible to effectively release hydrogen sulfide and/or sulfur-containing gas at a temperature of 100° C. or higher and 400°° C. or lower. Activated carbon also as a low level of decomposition in the temperature range of 100°° C. or higher and 400° C. or lower, and can therefore be repeatedly used as a hydrogen sulfide adsorbent, thus reducing costs for materials and for replacement.

The hydrogen sulfide adsorbent and the hydrogen sulfide removing agent may be the same or different.

### (Heating Section)

As illustrated in FIG. 8, the gas detection unit 140 may comprise a heating section 142 that heats the hydrogen sulfide adsorbent 141. The heating section may heat the hydrogen sulfide adsorbent at a temperature of 100° C. or higher and 400° C. or lower. The heating section is not particularly restricted so long as it can heat the hydrogen sulfide adsorbent, and it may be a heater, for example.

### (Gas Detection Section)

As illustrated in FIG. 8, the gas detection unit 140 may comprise a gas detection section 143 that detects hydrogen sulfide and/or sulfur-containing gas that has been released from the hydrogen sulfide adsorbent 141. Since heating of the hydrogen sulfide adsorbent will sometimes generate sulfur-containing gases other than hydrogen sulfide, such as sulfur oxides, the gas detection section may be one that can detect sulfur-containing gases that include hydrogen sulfide. The gas detection section is not particularly restricted so long as it can detect hydrogen sulfide and/or sulfur-containing gas. The gas detection section may be a gas sensor, for example.

When the purpose is to detect generation of hydrogen sulfide, such a construction results in contact of the gas detection section with highly concentrated hydrogen sulfide, thus allowing high-precision detection of hydrogen sulfide. Since the hydrogen sulfide released from the hydrogen sulfide adsorbent is removed by the hydrogen sulfide removing agent, this can prevent hydrogen sulfide from being emitted out of the vehicle.

### <Uses>

The battery pack of the disclosure can be used as a drive power supply for a vehicle. The vehicle is not particularly restricted and may be a hybrid vehicle (HEV), plug-in hybrid vehicle (PHEV), battery electric vehicle (BEV), gasoline automobile or diesel automobile, for example. A hybrid vehicle (HEV), plug-in hybrid vehicle (PHEV) or battery electric vehicle (BEV) is particularly preferred. The shape and size of the vehicle is not particularly restricted so long as it is able to have the battery pack installed.

### REFERENCE SIGNS LIST

10 Battery pack
110 Sulfide-based battery
120 Hydrogen sulfide removal unit
121 Hydrogen sulfide removing agent
122 Hydrogen sulfide removing agent container
122a Baffle plate
122b Distribution port
130 Outer container
131 Opening
140 Gas detection unit
141 Hydrogen sulfide adsorbent
142 Heating section
143 Gas detection section

1. A hydrogen sulfide removal unit, comprising a hydrogen sulfide removing agent and a hydrogen sulfide removing agent container housing the hydrogen sulfide removing agent, wherein:
the hydrogen sulfide removing agent container has at least one baffle plate, and
the baffle plate is disposed at an angle within 10° from the vertical direction so as to connect the top surface and bottom surface of the hydrogen sulfide removing agent container.

2. The hydrogen sulfide removal unit according to claim 1, wherein:
the perimeter of the baffle plate is joined to the inner peripheral surface of the hydrogen sulfide removing agent container,
the baffle plate at least partially has a distribution port, and
the ratio of the area of the distribution port with respect to the total area of the baffle plate and the distribution port is 30% or lower.

3. The hydrogen sulfide removal unit according to claim 2, wherein:
the hydrogen sulfide removing agent is disposed at least at the lower part inside the hydrogen sulfide removing agent container, and
the distribution port is provided at the lower part of the baffle plate.

4. The hydrogen sulfide removal unit according to claim 1, wherein the hydrogen sulfide removing agent container has a plurality of the baffle plates.

5. The hydrogen sulfide removal unit according to claim 4, wherein a meandering flow passage is formed by the plurality of baffle plates.

6. The hydrogen sulfide removal unit according to claim 2, wherein the hydrogen sulfide removing agent container is square cylindrical or rounded square cylindrical.

7. The hydrogen sulfide removal unit according to claim 6, wherein:
the hydrogen sulfide removing agent is disposed at least at the lower part inside the hydrogen sulfide removing agent container,
the distribution port is provided at the lower part of each baffle plate, and
the distribution port of the 2m+1-th baffle plate, where m is 0 or a positive integer, and the distribution port of the 2n-th baffle plate, where n is a positive integer, are provided so as to contact mutually different side surfaces among the pair of side surfaces of the hydrogen sulfide removing agent container, thereby forming the meandering flow passage.

8. The hydrogen sulfide removal unit according to claim 1, wherein each baffle plate has a thick part that is thicker than the other parts.

9. A battery pack, comprising a sulfide-based battery, a hydrogen sulfide removal unit according to claim 1, and an outer container housing the sulfide-based battery and the hydrogen sulfide removing agent, wherein:
the outer container has an opening allowing communication between the inside and outside of the outer container, and
the hydrogen sulfide removal unit is disposed so that the inside of the outer container can communicate with the outside of the outer container through the hydrogen sulfide removal unit and the opening.

10. The battery pack according to claim 9, wherein:
the battery pack further comprises a gas detection unit housed in the outer container,
the gas detection unit comprises a hydrogen sulfide adsorbent that adsorbs hydrogen sulfide and releases the hydrogen sulfide and/or sulfur-containing gas derived from the hydrogen sulfide at or above a predetermined temperature, and a heating section that heats the hydrogen sulfide adsorbent, and a gas detection section that detects the hydrogen sulfide and/or sulfur-containing gas released from the hydrogen sulfide adsorbent, and
the gas detection unit and the hydrogen sulfide removal unit are disposed so that the inside of the outer container can communicate with the outside of the battery pack through the gas detection unit, the hydrogen sulfide removal unit and the opening.
